(19) United States
(12) Patent Application Publication
(10) Pub. No.: US 2010/0070389 A1
(43) Pub. Date: Mar. 18, 2010

(54) METHOD FOR CLASSIFYING, IDENTIFYING AND FINDING AUTOMATICALLY, BY MEANS OF INFORMATIC DEVICES AND SYSTEMS, OF SURGICAL INSTRUMENTS OF VARIOUS KIND, AND FOR PROCESSING AND MANAGING DATA RELATING TO THE AUTOMATIC CLASSIFICATION, IDENTIFICATION AND FINDING OF SURGICAL INSTRUMENTS, OBTAINED BY MEANS OF SUCH INFORMATIC DEVICES AND SYSTEMS

(76) Inventor: Juan Farabola, Urbano (IT)

Correspondence Address: BUCKNAM AND ARCHER, 1077 NORTHERN BOULEVARD, ROSLYN, NY, 11576, US

(21) Appl. No.: 12/312,803

(22) PCT Filed: Mar. 5, 2007
(86) PCT No.: PCT/IB2007/000569

§ 371 Date: May 27, 2009

(30) Foreign Application Priority Data
Nov. 28, 2006 (IT) PN2006A000098

(51) Int. Cl. G06Q 10/00

(52) U.S. Cl. 705/28; 705/2

(57) ABSTRACT

A method for classifying, identifying and finding automatically, by means of informatic devices and systems, particularly personal computers, of surgical instruments of various kind employed in sanitary structures for performing surgical operations. The method includes the following steps:

preparation, by the specialized firm for performing such repair, maintenance or substitution interventions of the surgical instruments, of a first finding card with the data including the names of the containers of these instruments and of the same instruments, a second signalling card of the surgical instruments with the data of the names of all the defective instruments and the technical interventions to be effected thereon, a third re-composition card with the data of the names of all the surgical instruments to be used for each surgical operation, a fourth card with the data of the matrixes, markings and identifying plates of the different types of surgical instruments, for finding them, and a fifth container card, with the data of the names of all the containers;
inputting and storing all the data contained into said first, second, third, fourth and fifth card into said informatic devices and systems, and processing and managing the same data for the automatic classification, identification and finding of all the available surgical instruments which can be used by each sanitary structure.

The invention relates to a method for classifying, identifying and finding automatically, by means of informatic devices and systems, of surgical instruments of various kind, and for processing and managing data relating to the automatic classification, identification and finding of surgical instruments, obtained by means of such informatic devices and systems. At the present time, in the surgical field employing surgical instruments of various kind for a plurality of operations, the manufacturing firms of these instruments for sanitary uses provide for classifying and identifying the same with standardized wordings and signs, which are specific for each firm and identify the name of the manufacturing firm of the type and quantity of the manufactured and offered products and these classifications are used also by the distributors of these products and by the various sanitary structures (hospitals, nursing-homes, ambulatories, university hospitals etc.), which buy and use the same instruments, which provide for reporting the names of the various surgical instruments which have been bought into cards, forms, files etc. having paper and/or electronic formats, in this latter case into personal computers in which such data are stored and made available and accessible for the employed sanitary staff. In turn, the surgical instruments are put and kept into special cupboards or other suitable case means of the respective sanitary structures, from which they may be found and, when their use is requested, are picked up by the respective operating-theatres, where these instruments are used and, after their use, are put again into such containers, where they can be picked up again, washed and sterilized before a subsequent use thereof, or can be picked up for repairing, maintaining, substituting operations etc.

These managing manners of the surgical instruments do not allow the single sanitary structures to know and recognize with safety the types, quantities and manufacturers of the surgical instruments actually available by the same structures, and do not allow to find with safety the single or total instruments when they are needed, after all for the rinsing, washing and sterilizing operations for being employed on the operating-theatres, and also for repair, maintenance or substitution operations thereof, which are needed for the availability of perfectly efficient instruments, and such incapacity of finding the instruments involves consequently considerable difficulties for the employed staff, as far as the knowledge of the up-to-dated situation of the types, quantities and preservation state and operative capacity of these instruments is concerned, thereby creating an uncertainty situation which may cause wrong valuations and initiatives on the matter by the employed staff.

The object of the present invention is to eliminate the drawbacks and troubles deriving from the above mentioned manners of classifying and identifying the surgical instruments, which do not allow them to be found by the different sanitary structures for the grounds described as above, and this is obtained according to the invention by means of a new and original method for classifying, identifying and finding of the various employed instruments, which method is utilized in combination with electronic informatic devices and systems, and may be used and made compatible with all the existing sanitary structures. Moreover, such method allows in a simple, easy and quick way to process and manage by the used informatic devices and systems data relating to such classification, identification and finding of the surgical instruments available on each sanitary structure.

This method has the characteristics and is composed of the operative steps substantially described with particular reference to the attached claims of the present patent.

The invention will be better understood by the following description, given by way of not-limitative example only, and with reference to the accompanying drawings wherein:

FIG. 1 shows a finding card according to the invention, prepared with all the requested data for identifying with safety all the available surgical instruments to be used, and for knowing where they are kept and can be found with safety, as well as for knowing the operative efficiency state thereof;

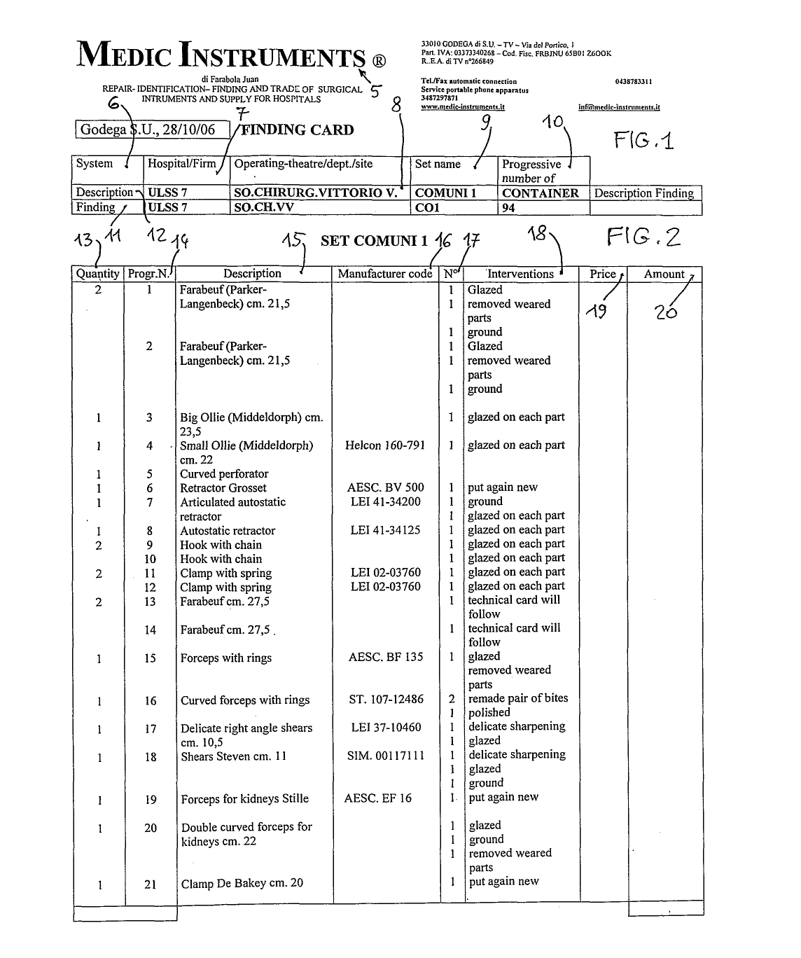

FIG. 2 shows an example of a finding card according to the invention, prepared with all the requested identifying data for finding with safety all the surgical instruments arranged into a specific container, and the interventions performed on to the same instruments which are listed in detail for each instrument, for complying with the international warranty specifications;

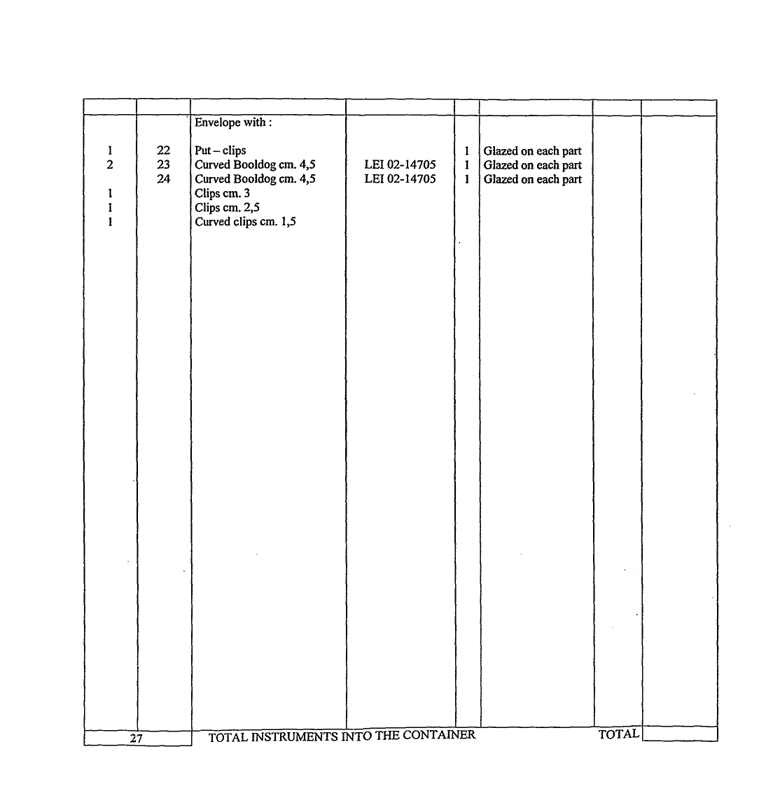

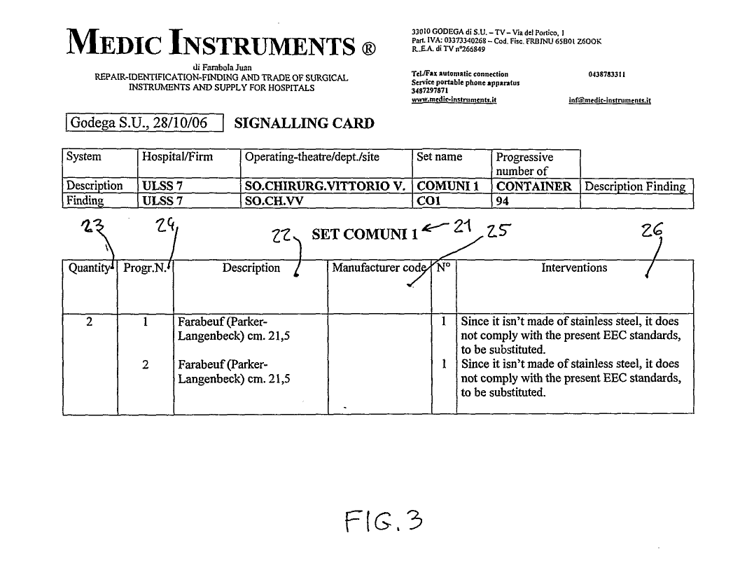

FIG. 3 shows an example of a signalling card according to the invention, in which all the surgical instruments being surveyed are indicated, relative to the operative efficiency and the respective valuations thereof;

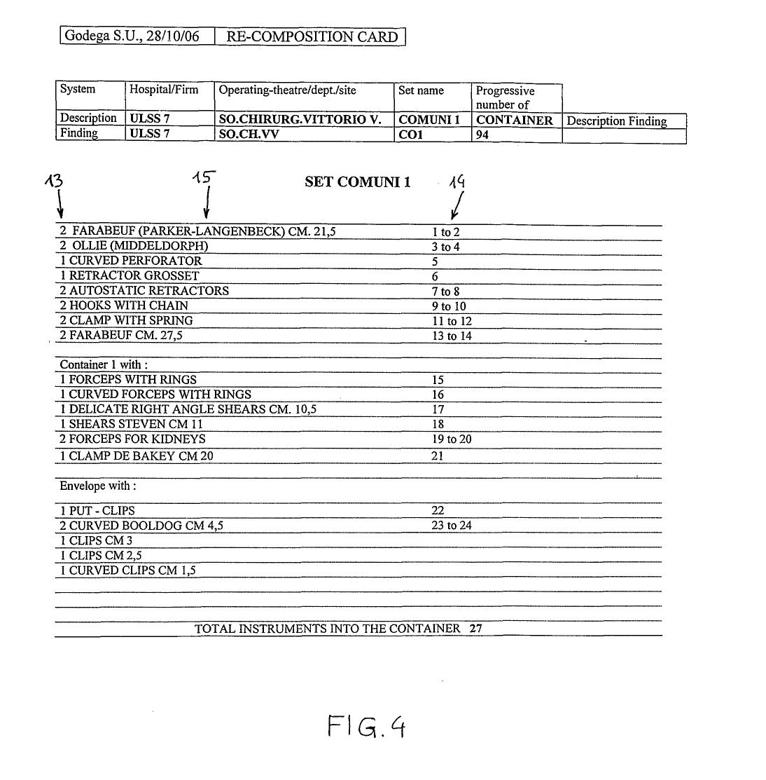

FIG. 4 shows an example of a re-composition card according to the invention, in which all the surgical instruments being of perfectly operative efficiency are indicated, and which may be arranged into a specific container and made available for use in a relative operating-theatre;

FIG. 5 shows an example of a card for matrixes, markings and plates for finding, in which all the surgical instruments to be marked are indicated, together with the relative plates for finding thereof, which are arranged into a determinate container;

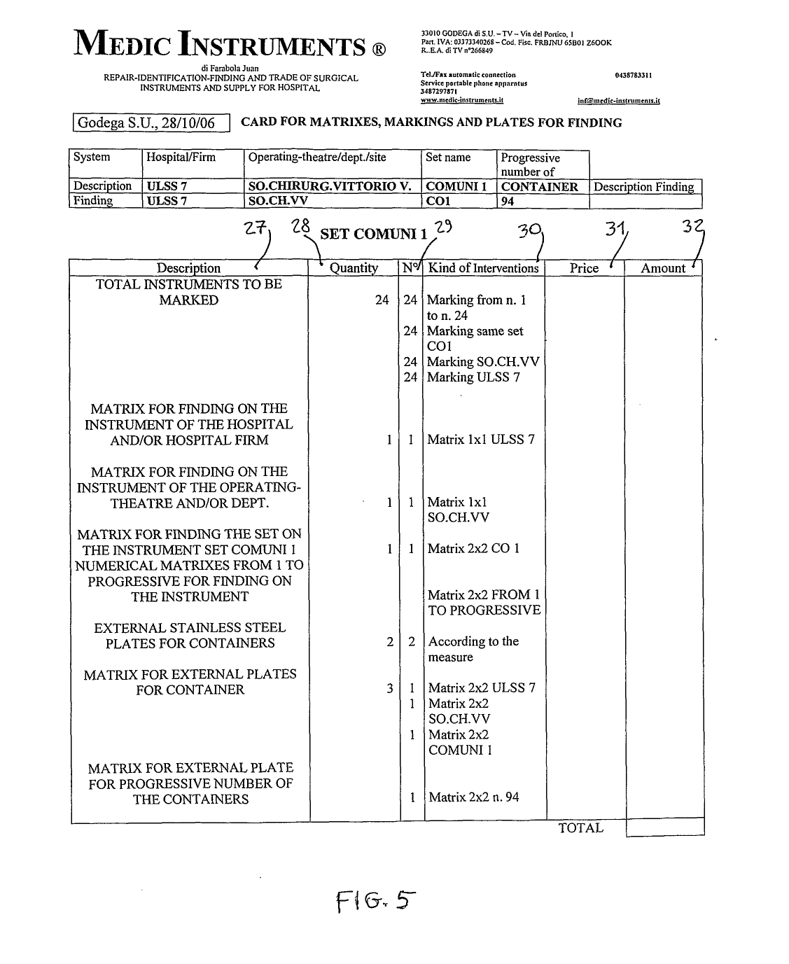

FIG. 6 shows an example of a container card according to the invention, in which all the containers with the relative date of the last performed maintenance are indicated, which are all pertaining to the same hospital and each operating-theatre;

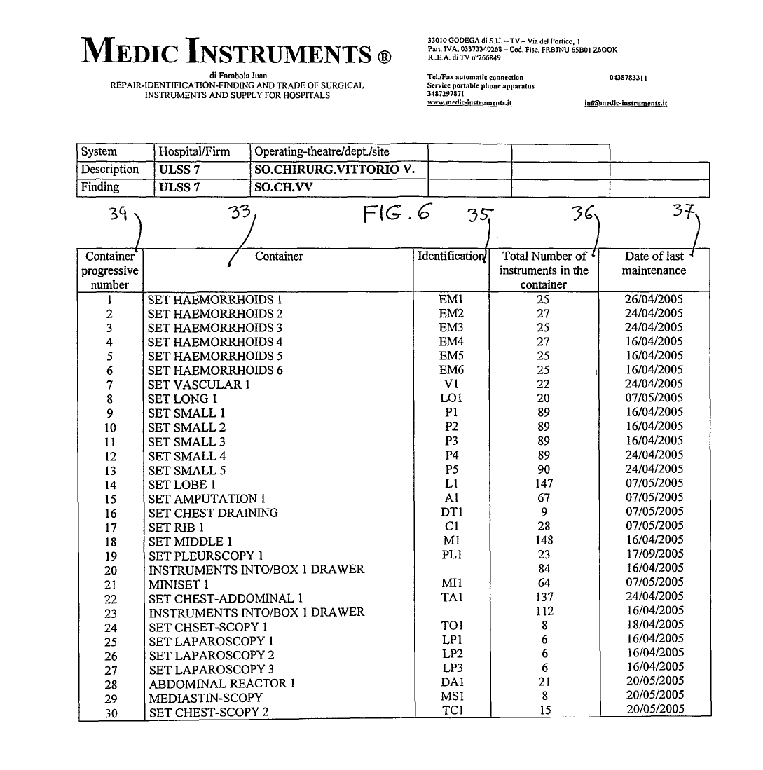

FIG. 7 shows an example of an identification plate for surgical instruments containers, with the classification and identification according to the invention;

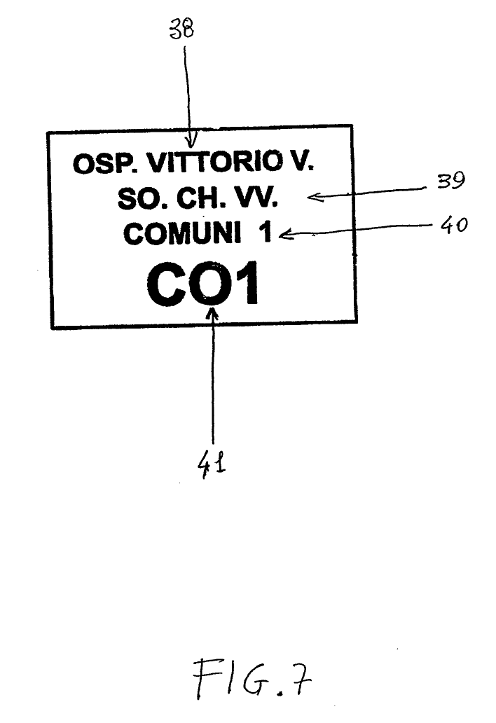

FIGS. 8 and 9 show a respective front and back view of a type of used surgical instrument, which is marked on its front and back surfaces with abbreviated identifying, classifying and finding wordings, in accordance with the method of the present invention;

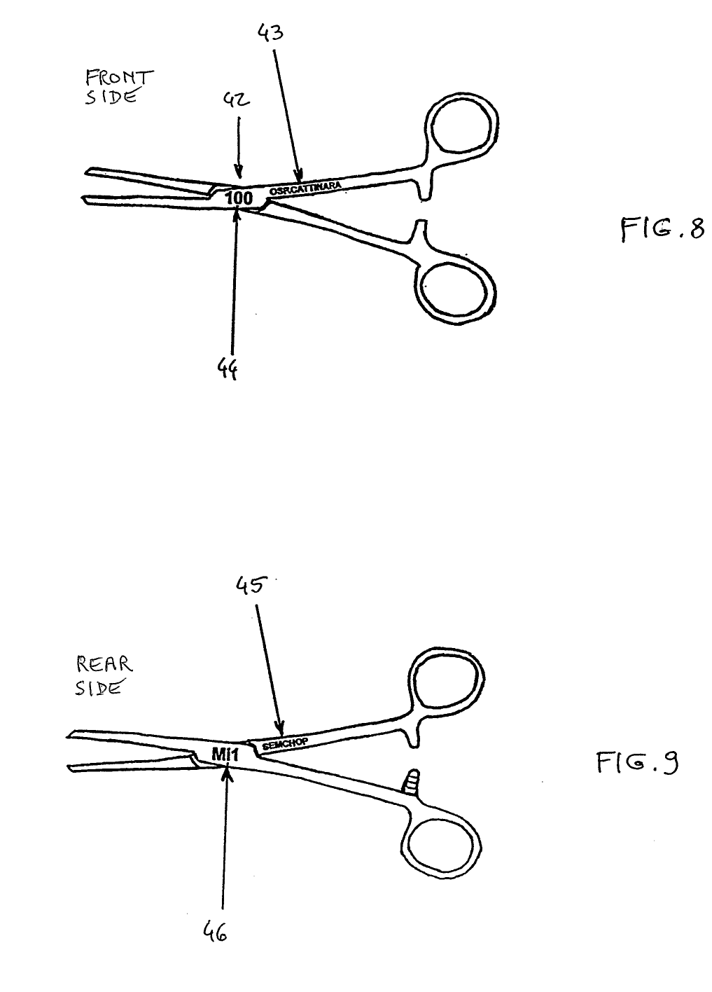

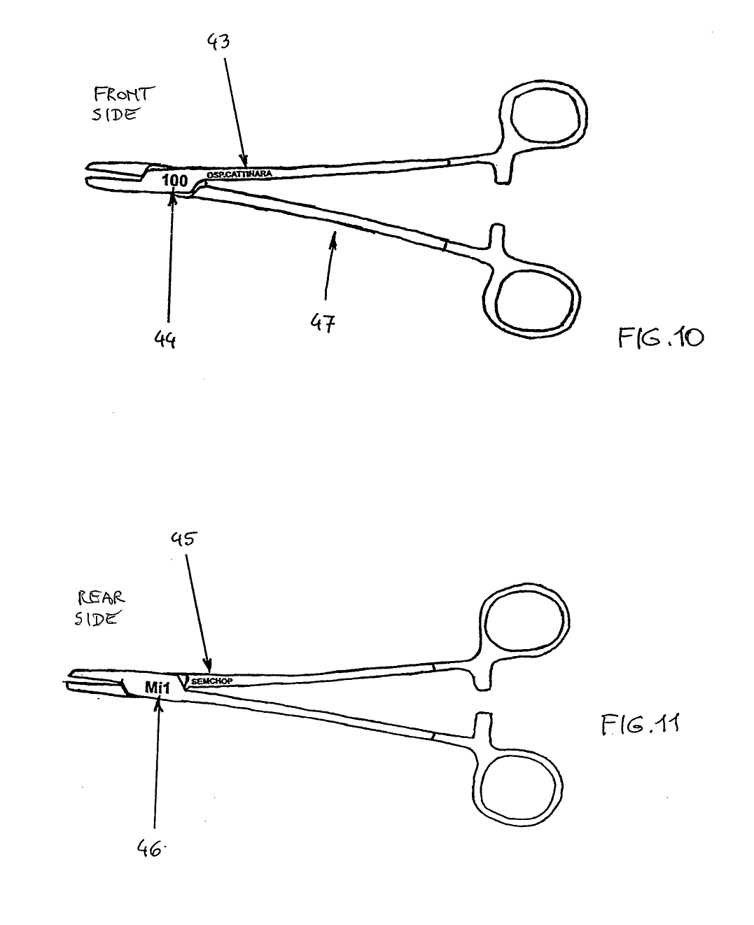

FIGS. 10 and 11 show a respective front and back view of another type of used surgical instrument, marked like the FIGS. 8 and 9, in accordance with the present method;

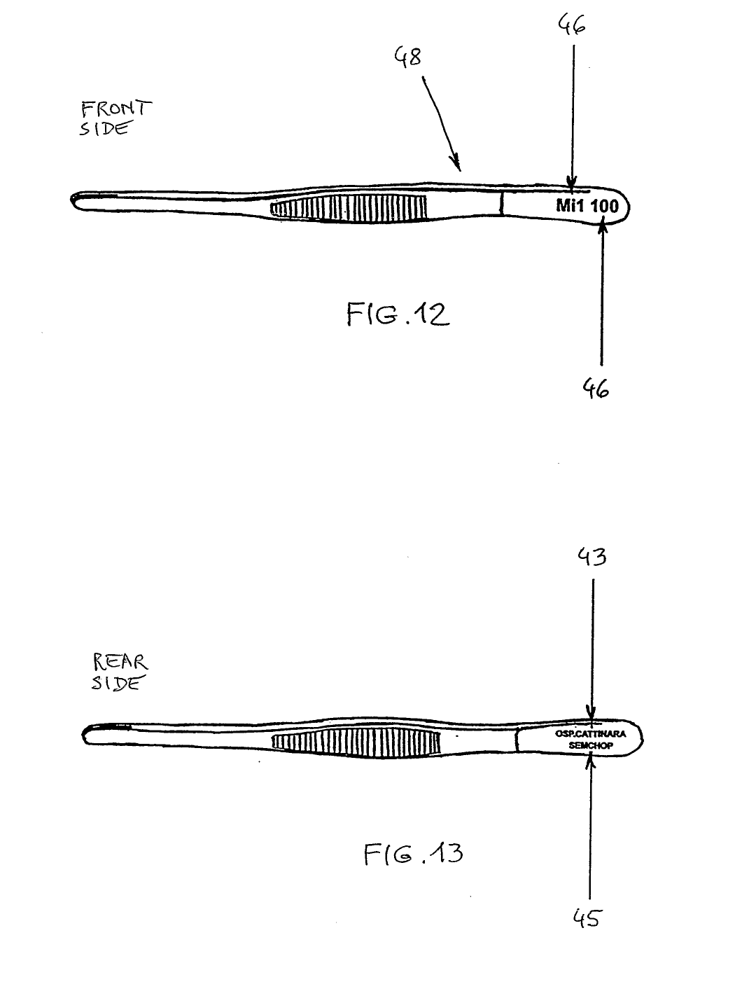

FIGS. 12 and 13 show a respective front and back view of a further type of used surgical instrument, marked like in the FIGS. 8, 9, 10 and 11, in accordance with the present method.

The present invention relates to a method for classifying, identifying and finding automatically, by means of informatic devices and systems of various kind, such as for example computers and microchips; of surgical instruments of various kind which are used for performing operations in the various existing sanitary structures like hospitals, nursing-homes, departments, ambulatories, university hospitals etc.

Such method also relates to the processing and managing of data concerning to the automatic classification, identification and finding of surgical instruments, obtained by means of such informatic devices and systems. The identification is also “loaded” onto at least a microchip, welded to the relative instrument and container having a suitable containing housing made of stainless steel. The container microchip has loaded onto itself the totality of instruments and, by means of an on-off switch, offers the safety that all the instruments have been put into the relative container. The method referred to consists essentially in the preparation of a set of cards, which will be described in detail further on, in which all the data concerning the surgical instruments relative to the classification, identification and finding of the surgical instruments available by the sanitary structures are reported. The data contained into these cards are then introduced and stored into the informatic devices and systems used by such sanitary structures, and are then called back when needed and up-to-dated with further substitute or supplementary data concerning any type of change relative to the surgical instruments, as it will be described in detail later on.

According to the method of the invention, the cards which are prepared and filled in are the following:

1) Finding card (see FIG. 1)—this card includes the following data:
1 a) the indication of the name and address of the firm devoted to effect the needed interventions on to the different surgical instruments available by a determinate sanitary structure, marked with the reference numeral 5, in this example for signalling the interventions effected for repairing, maintaining and/or substituting determinate surgical instruments available by such sanitary structure. Moreover, the date in which the card is filled in.

In the example referred to, the interventions have been effected by the Applicant of this patent application;

1 b) a list containing the following data:
1 c) the indication of the generic system namely the sanitary structure, marked with the reference numeral 6;
1 d) the specific indication of the type of sanitary structure, in the example hospital or firm, marked with the reference numeral 7, utilizing the surgical instruments which are put into specific containers employed at this time, and taken by them, which instruments have been already submitted to various interventions;
1 e) the indication of the operating-theatre/department devoted in this matter and pertaining to the site of the sanitary structure, indicated by 8;
1 f) the indication of the name of the container/set used according to the type of operation, for taking the treated surgical instruments, marked with the reference numeral 9;
1 g) the progressive number given to each used container/set, marked with the reference numeral 10;
1 h) the description, marked with the reference numeral 11, of all the specific data under the points 1 d), 1 e), 1 f), 1 g);
1 i) the exact and complete name of the sanitary structure referred to the point 1 d);
1 l) the exact and complete name of the operating-theatre/department/site under the point 1 e);
1 m) the specific and complete name attributed to each container/set in which the surgical instruments are put and taken;
1 n) the date of the maintenance effected previously of all the surgical instruments put and taken in the container referred to;
1 o) the finding, marked with the reference numeral 12, of all the specific data under the points 1 d), 1 e), 1 f), 1 g). According to the method of the invention, all the data relative to the point 1 d) are formed by the initial letters of the relative sanitary structure and the complete or partial name thereof, while all the data relative to the point 1 e) are formed by the initial letters of the operating-theatre/department/site indicated under the point 1 d). In turn, all the data relative to the point 1 f) are formed by alphabetical-numerical initials composed of letters and a progressive number, which are codified in an appropriate manner and indicated on specific lists, which are made available to the employed staff of the sanitary structure and the firms co-operating therewith.

In the example indicated on FIG. 1, the different data reported on the finding card are marked as follows:
name of the sanitary structure under the point 1 i): ULSS 7,
name of the operating-theatre/department/site indicated under the point 1 l): so. Surgical Vittorio V.;
specific and complete name of each container under the point 1 m): COMUNI 1;
date of the preceding maintenance under the point 1 n):
indication of the sanitary structure: ULSS 7;
indication of the operating-theatre/department/site: SO.CH.VV;
indication of the employed container/set: CO 1;
progressive number of the container: 94.

In the so arranged finding card there are then reported the complete name of type of employed container/set for keeping the requested surgical instruments, see FIG. 2, and the interventions effected by the firm devoted to repair, maintain or substitute the same instruments, which in this case is always the Applicant of the present patent application, and such container/set is constituted by the container/set named SET COMUNI 1.

The indications relative to the surgical instruments put into the employed container are the following:

2 a) The whole quantity of each employed surgical instrument, marked with the reference numeral 13;
2 b) the progressive number of each employed surgical instrument, which is marked on to the relative instrument as it will be described later on, without using bar codes, thereby permitting a quick identification thereof by any person, also by the persons not specialized in the matter, such number being marked with the reference numeral 14;
2 c) the description of the type of employed surgical instruments, having standard names which are used and supplied by any manufacturing firm of these instruments and which are used also by the distributors of such firms and by the sanitary structures which buy and use the same instruments, such description being marked with the reference numeral 15;
2 d) the reference of the code of the surgical instruments manufacturer, which is prepared and used by each single manufacturing firm of these instruments and which is used also by the distributors of these firms and by the sanitary structures which buy and use the same instruments, such reference being marked with the reference numeral 16;
2 e) the number of maintenance interventions effected for each surgical instrument, marked with the reference numeral 17;
2 f) the interventions effected for each surgical instrument, and the succinct description thereof, marked with the reference numeral 18;
2 g) the price and the amount of the costs debited to the sanitary structure for each intervention effected on the surgical instruments, marked with the respective reference numerals 19 and 20.

In the example reported in the card illustrated by the FIG. 2, in which there are indicated the specific types of the different surgical instruments put into the container/set and the needed interventions effected for the same with the relative costs, it is thus possible to have a complete situation relative to such instruments, permitting to find quickly and without possibility of mistakes each instrument being in perfect operative efficiency, which is available by each sanitary structure.

The terminal part of this card reports the whole number of the surgical instruments contained into the container/set and the total amount of the costs for all the interventions effected for the same instruments.

Preferably, the finding cards illustrated and described by FIGS. 1 and 2 are included for convenience and better clearness into a single card, which is prepared and filled in by the firm which has effected the required interventions on the surgical instruments, and is made available by the same for the sanitary structure, which so may know the exact up-to-dated situation of all the treated surgical instruments.

By referring now to the FIG. 3, shown therein is a signalling card according to the invention, with the indications, prepared by the firm effecting the required interventions on the surgical instruments put into each container/set, of some surgical instruments being surveyed relative to their operative efficiency and the relative technical valuations thereof. As visible, this signalling card includes at its upper part all the indications relative to finding thereof, indicated under point 1, and also includes the following data:

3 a) the indication of the name of the container/set containing the surgical instruments, marked with the reference numeral 21. In the example referred to, such indication concerns the set COMUNI 1.

Then, the description of the examined surgical instruments is supplied, which is preceded by the quantity and the progressive number marked on the same instruments, and is followed by the code of the manufacturer of such instruments, and by the relative detailed indication of the technical interventions to be effected, in this case consisting of the description of the technical defects which have been found and the need to substitute these instruments with further identical and new instruments. These items are marked with the respective reference numerals 22, 23, 24, 25 and 26.

By referring now to the FIG. 4, shown therein is a re-composition card according to the invention, in which there are indicated all the surgical instruments which must be used for a determinate operation in the operating-theatre, which instruments are all in perfect operative efficiency, even after a possible repair and/or maintenance by the devoted firm, and for this purpose they are picked up by the employed staff from the cupboard or other holding site thereof and put into a defined container/set, which is submitted in succession to rinsing, washing, sterilizing and drying operations with conventional methods, and thereafter such container/set with all the perfectly efficient instruments is taken into the operating-theatre, where the instruments are picked up and made available for the surgeons. At the end of the operation, the instruments are cleaned carefully and still put into the container/set, for being so brought back into the cupboard or other holding site thereof. Therefore, the function of this card is that to know exactly and in detail all the surgical instruments which will be employed and the re-composition thereof into the container/set, with the absolute safety that all these instruments are in a perfect efficiency state, thanks to the fact to identify with safety each instrument being employed, and to know exactly that the defective and/or inefficient instruments have been adequately treated and/or substituted, by making them perfectly efficient thanks to the use of the finding card. In this way, thanks to the use of the re-composition card, which identifies also the used container/set for taking the surgical instruments, it is excluded with absolute safety by the employed staff of the sanitary structure any mistake possibility of the re-composition of the instruments into the used and identified container/set, or of exchange of the required instruments with other not needed instruments, so as to ensure also that all the instruments are perfectly efficient and submitted certainly to the needed sterilization treatments before each operation. Each re-composition card contains at first on its upper part all the indications relating to finding thereof, indicated under the point 1).

Thereafter, this card contains the indication of the name of the used container/set, as reported by the FIG. 2, and this type of container in the example illustrated by the FIG. 4 is marked with the name SET COMUNI 1.

Hereinafter, in the card referred to there are reported all the types of used instruments and put into this type of container, which are marked as described under the point 2 c) and are preceded and followed respectively by the quantity and the progressive number of each type of used instruments. Finally, on the underlying area of this card it is indicated the total number of the instruments put into the container/set.

By referring now to the FIG. 5, shown therein is a card for matrixes, markings and plates which are used for finding the instruments used for a determinate operation and put into and taken with a specific container/set. This card contains on its upper part all the indications concerning finding thereof, which are indicated under the point 1, and also contains the following data:

5 a) the description of the total surgical instruments to be marked and the relative used marking equipment, marked with the reference numeral 27,
5 b) the total quantity of the surgical instruments and the relative marking equipment with the relative part number, the type of intervention to be effected, the price and amount to be paid, marked with the respective reference numerals 28, 29, 30 and 31.

Besides, on the card lower part it is indicated the total amount of the costs of the effected markings.

By referring now to the FIG. 6, shown therein is a container card according to the invention, in which there are indicated all the surgical instruments contained into a plurality of specific containers, with the relative dates of the last maintenance effected thereon. As usual, each card contains on its upper part all the indications relative finding of the different used containers/set, described under the point 1.

Moreover, the card contains also the name of the container/set containing the instruments indicated in the finding card, and the abbreviated indication of the same name, which are codified in an appropriate manner by the firm performing the interventions on the instruments, and are indicated into adequate lists, which are made available for the employed staff of the sanitary structure and the firms co-operating therewith.

In particular, the codified name of the containers/set reported into this card is marked with the reference numeral 33 and is preceded by the progressive number of such containers/set, marked with the reference numeral 34, and is followed by the abbreviated identification code of the relative name, the number of instruments contained into the relative containers/set, and the date of the last maintenance, which are marked with the respective reference numerals 35, 36 and 37. In this way, this card allows to know and find quickly all the containers/set in which there are put all the surgical instruments to be used for the specific surgical operations in the relative operating-theatres, and for complying with the sterilization rules, which instruments are found and identified with the relative re-composition cards, thereby excluding with absolute safety by the employed staff any possibility of mistakes for finding the containers/set, or to exchange the containers/set with other not needed containers/set, in the operating-theatres and the washing and sterilizing units. By referring now to the FIG. 7, shown therein is an example of an identifying plate for finding in the containers/set the surgical instruments which are utilized by the sanitary structures. Such plate contains a set of identifying writings, which are engraved thereon, and which contain the writings indicated under the point 1, as follows:

7 a) name of the hospital or hospital firm, with the abbreviated identifying code, marked with the reference numeral 38;
7 b) name of the operating-theatre/department/site, with the abbreviated identifying code, marked with the reference numeral 39;
7 c) name of the container/set as indicated into the container card, with the relative abbreviated identifying code, indicated respectively with the reference numerals 40 and 41, followed by the progressive number of the container pertaining to each sanitary structure. The so composed identifying plate is applied on to the outer part of the relative containers/set and also on to the inner part of the same containers, in this case without the name under the point 7 c).

By referring now to the FIGS. 8-13, shown therein are the markings which are affixed by means of electrolytic marking on to the front and back surfaces of some types of used surgical instruments. In particular, the FIGS. 8 and 9 show a ring pincers 42, the front surface of which is marked with the name of the pertaining sanitary structure and the progressive number of each instrument, marked with the respective reference numerals 43 and 44, and the back surface of which is marked with the name of the operating-theatre/department/site of such sanitary structure, its identifying code as well as the name of the relative container together with its abbreviated identifying code, indicated respectively with the reference numerals 45 and 46, followed by the progressive number of the container. Furthermore, FIGS. 10 and 11 show the markings affixed with the same criteria on to another type of ring pincers 47, while FIGS. 12 and 13 show the markings affixed with the same criteria on to a standard pincers 48. In this way, the so marked and identified instruments may be found with absolute safety and without possibility of mistakes by the employed staff of the sanitary structure, in the site in which they are kept when their use isn't requested, when they must be submitted to interventions of various kind, and when they need to be employed in the operating-theatre and when they are taken in the washing and sterilizing units. According to the present method, the above mentioned cards are also inputted and stored, with the relevant identification and finding data of the used surgical instruments, into informatic devices and systems of various kind such as for example personal computers, in order to process and manage all the data relating to the automatic classification, identification and finding of the surgical instruments available by the sanitary structures, which data are made available through the cards referred to and any possible microchips included into the surgical instruments and the containers.

Thus, with the criteria of this method it is possible, for the firm devoted to effect interventions of repair, maintenance and/or substitutions of the surgical instruments available by the sanitary structures, to inspect carefully these same instruments and to effect particular interventions on to the same, to render them perfectly efficient in their operation and give them back to the relative sanitary structure for the use thereof on the conditions of greatest safety and without mistakes by the employed staff of the same sanitary structure. First of all, all the surgical instruments to be inspected are introduced into one or more containers of the sanitary structures and made available for the interested firm, together with a list of all the delivered instruments, prepared by such interested firm, in a way that this firm may check if the instruments contained into the container correspond to the list received by the sanitary structure, thereby noticing the actual correspondence or any difference or absence of the instruments contained into the container with respect to those reported into the list.

Under these conditions, the present method foresees the carrying out of the following steps:
annotation on the finding card of all the delivered surgical instruments and any difference or absence thereof with respect to those reported into the list, these latter being in case noted down also in the signalling card;
appraisal on the inspection bench of each single type of surgical instrument, to know the maintenance interventions to be effected for each one of them, or which instruments need to be substituted, and annotation of these interventions into the finding card.

All the effected maintenance interventions are then noted down always into this finding card, with the relative maintenance date which is reported under the point 1 n;
annotation on the signalling card of all the instruments which need to be substituted;
application to all the types of the containers/set supplied by the sanitary structure for containing the surgical instruments of the relative markings and plates for finding the same containers/set, and annotation of all the markings and plates into the card for matrixes, markings and plates for finding;
annotation in each re-composition card of a specific container/set, identified as described under the previous point, of all the surgical instruments which can be found from the finding card and which must be employed into a determinate operating-theatre, in order to submit all the instruments to the needed washing and sterilizing treatments before the foreseen operation, such instruments being picked up, after the operation has been accomplished, by the employed staff and taken to the sterilizing unit, where they are washed, re-composed into the containers and sterilized, for being subsequently put into the housing places and, according to the needs, re-delivered to the operating-theatres. The instruments are extracted from their own sterile container and put on to stainless trays, with engraved the required composition thereof for the operation according to the progressive numbering and the other finding wordings, in accordance to the surgeon needs or habits.

It is also to point out that the microchips used for identifying the instruments and containers may be applied to these latter not only by means of welding, as previously mentioned, but also by providing relative housings by means of milling, into which the same microchips are arranged, and then by covering the whole with an appropriate plug with steel cylinder. Therefore, the used informatic device or system having the cards of the kinds referred to already inputted and stored, makes extremely easier to effect all the above described various steps of the present method.

1. Method for classifying, identifying and finding automatically, by means of informatic devices and systems, particularly personal computers and microchips, of surgical instruments of various kind employed on sanitary structures for performing surgical operations, which are normally kept into cupboards or other adequate housing sites, which are picked up and kept into appropriate containers, for being taken toward the relative operating-theatres, before having been washed and sterilized by per se known means, and after each operation they are cleaned and taken toward the relative housing sites, and these instruments are also taken toward specialized firms for performing repair, maintenance or substitution interventions of the same instruments, and when such interventions are performed they are made available again by these firms for the sanitary structures, characterized by the following steps:
preparation, by the specialized firm for performing such repair, maintenance or substitution interventions of the surgical instruments, of a first finding card with the data including the names of the containers of these instruments and of the same instruments, which are brought on a condition of perfect operative efficiency after that the needed interventions have been performed; a second signalling card of the surgical instruments with the data of the names of all the defective instruments for the substitution, by reporting the technical causes or the breakages thereof; a third re-composition card with the data of the names of all the surgical instruments to be used for each surgical operation, included those submitted to the repair, maintenance or substitution interventions, and the relative containers where these instruments are put; a fourth card with the data of the matrixes, markings and identifying plates of the different types of surgical instruments, for finding them; and a fifth container card, with the data of the names of all the containers;
inputting and storing all the data contained into said first, second, third, fourth and fifth card into said informatic devices and systems and microchips for the remote recognition and survey, and processing and managing the same data for the automatic classification, identification and finding of all the available surgical instruments which can be used by each sanitary structure.

2. Method according to claim 1, characterized in that said first, second, third, fourth and fifth card include in common the following data:
indication of the name and address of said specialized firm,
indication of the system namely the generic sanitary structure (6), with the specific indication of the type of sanitary structure (for example, hospital or firm 7), and the name of the same sanitary structure, included the coded name of this latter, for finding thereof;
indication of the operating-theatre/department/site devoted in the matter (8) and pertaining to this type of sanitary structure, with the indication of the name of this operating-theatre/department/site, included the coded name thereof, for finding them,
indication of the name of the utilized container/set (9) for taking the surgical instruments, with the indication of the name of this container/set and the coded name thereof, for finding it,
the progressive number attributed to each employed container/set (10).

3. Method according to claim 2, characterized in that said finding card also includes:
the total quantity (13) of each type of used surgical instrument,
the progressive number of each used surgical instrument (14),
the description of each type of used surgical instrument (15), which has been identified and found with the data contained into said fourth card and formed with standard names which are used and supplied by each single manufacturing firm of these instruments and which are also used by the distributors of these firms and by the sanitary structures buying and using the same instruments,
the reference (16) to the code of the manufacturer of the surgical instruments, which is prepared and used by each single manufacturing firm of these instruments and which is also used by the distributors of these firms and by the sanitary structures buying and using the same instruments,
the number of intervention (17) for each single surgical instrument,
the effected interventions (18) for each single surgical instrument and the succinct description thereof,
the price and costs amount. (19, 20) debited to the sanitary structure for each effected 10 intervention on the surgical instruments, with the total costs amount for all the effected interventions.

4. Method according to claim 2, characterized in that said second signalling card also contains the following data:
indication of the name of the container/set (21) with the description (22) of the surgical 15 instruments which have been found defective and/or requiring the substitution, such description being preceded by the quantities (23) and the progressive number (24) marked on to the same instruments, and followed by the manufacturer code (25) of these instruments and the relative detailed indication (26) of the technical interventions to be performed thereon.

5. Method according to claim 3, characterized in that said re-composition card also contains the following data:
indication of the name of the used container/set for a determinate surgical operation,
indication of all the types of used surgical instruments (15) and put into such container/set, which indication is preceded and followed respectively by the quantity (13) 25 and the progressive number (14) of each type of used surgical instrument, with the final indication of the total number of used instruments.

6. Method according to claim 2, characterized in that said fourth card of the matrixes, markings and plates also contains the following data:
description (27) of the total surgical instruments to be marked and the relative marking 5 equipment,
the total quantity (28) of the surgical instruments and the relative marking equipment, with the relative number of parts (29), the type of intervention to be effected (30), the price (31) and the amount to be paid (32), with the total costs amount of the effected markings.

7. Method according to claim 2, characterized in that said fifth container card also contains the following data:
coded name (33) of the used containers/set and containing the surgical instruments to be used, preceded by the progressive number (34) of such containers/set and followed by the abbreviated identifying code (35) of the relative name,
number of instruments (36) contained into the relative containers/set, —date (37) of the last maintenance effected on the same instruments.

8. Method according to claim 7, characterized in that each identifying plate for finding said used containers/set contains the following identifying writings, which are engraved thereon:
name of the sanitary structure, with the abbreviated identifying code (38),
name of the operating-theatre/department/site, with the abbreviated identifying code (39),
name of the container/set (40), with the relative abbreviated identifying code (41), followed by the progressive number of the container/set pertaining to each sanitary structure.

9. Method according to claim 8, characterized in that each type of used surgical instrument is marked, on a side thereof, with the name of the pertaining sanitary structure (43) and the instrument progressive number (44), and on the other side thereof with the name (45) of the operating-theatre/department/site of this sanitary structure, with its abbreviated identifying code and the name (46) of the relative container with its abbreviated identifying code, followed by the progressive number of the same container.

10. Method according to claim 9, characterized in that each instrument is also marked with its pertaining data, which can be remotely detected by said microchips, applied on to the relative containers, and by appropriate detecting appliances.
